(19) United States
(12) Patent Application Publication
(10) Pub. No.: US 2016/0122797 A1
(43) Pub. Date: May 5, 2016

(54) MAGNETIC SEPARATION PROCESS USING CARBOXYL-FUNCTIONALIZED SUPERPARAMAGNETIC NANOCLUSTERS

(71) Applicant: 3M INNOVATIVE PROPERTIES COMPANY, Saint Paul, MN (US)

(72) Inventors: Manjiri T. Kshirsagar, Woodbury, MN (US); Lijun Zu, Woodbury, MN (US)

(21) Appl. No.: 14/895,993

(22) PCT Filed: Jun. 5, 2014
(86) PCT No.: PCT/US2014/041016

§ 371 Date: Dec. 4, 2015

Related U.S. Application Data
(60) Provisional application No. 61/833,545, filed on Jun. 11, 2013.

(51) Int. Cl. C12Q 1/10; C12Q 1/04

(52) U.S. Cl. CPC C12Q 1/10; C12Q 1/04

(57) ABSTRACT

A process including: contacting a plurality of carboxyl-functionalized superparamagnetic nanoclusters with a liquid sample potentially comprising at least one microorganism strain; magnetically separating at least some of the carboxyl-functionalized superparamagnetic nanoclusters from at least a portion of the liquid sample; and, assaying the magnetically-separated superparamagnetic nanoclusters for evidence of the at least one microorganism strain having been non-specifically bound thereto.

### BACKGROUND

It is often desirable to assay for the presence of bacteria or other microorganisms in various clinical, food, environmental, or other samples.

### SUMMARY

In broad summary, herein is disclosed a process comprising: contacting a plurality of carboxyl-functionalized superparamagnetic nanoclusters with a liquid sample potentially comprising at least one microorganism strain; magnetically separating at least some of the carboxyl-functionalized superparamagnetic nanoclusters from at least a portion of the liquid sample; and, assaying the magnetically-separated superparamagnetic nanoclusters for evidence of the at least one microorganism strain having been non-specifically bound thereto.

### DETAILED DESCRIPTION

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match. By diameter is meant the diameter of a spherical body; or, for an irregular body, the diameter of a sphere with the same volume as the irregular body. Terms such as (meth)acrylic, (meth)acrylate and so on, encompass both the acrylate and methacrylate version of the item referred to.

Herein is disclosed a process for separating at least one microorganism strain from a liquid sample in which the microorganism strain(s) may be present. The process relies on superparamagnetic nanoclusters that comprise carboxyl functional groups on the surfaces thereof, which carboxyl functional groups can non-specifically bind (e.g., individually, and/or in groups of two, three, four, or more, etc.) to the microorganism(s) if present. The superparamagnetic nanoclusters can then be magnetically separated from the liquid sample. After the superparamagnetic nanoclusters (potentially bearing microorganisms non-specifically bound thereto) are separated from the liquid sample, the nanoclusters may be assayed for evidence of the at least one microorganism strain having been non-specifically bound thereto.

By superparamagnetic is meant ferromagnetic or ferrimagnetic materials comprised of primary nanoparticles whose primary particle size is smaller than the single (magnetic) domain limit (e.g., around 30 nanometers for magnetite). In the presence of an externally applied magnetic field, such particles can display a magnetic susceptibility that is much higher than that of conventional paramagnetic materials. However (because of their extremely small primary particle size), in the absence of an externally applied magnetic field, thermal effects can overwhelm any magnetic effects so that the particles exhibit no overall permanent magnetic properties. That is, upon removal of an external magnetic field, a superparamagnetic material does not exhibit any permanent-magnet properties (as would be exhibited by e.g. larger-sized particles of e.g. ferromagnetic material). Superparamagnetic materials may be e.g. Fe₂O₃, Fe₃O₄ (magnetite), Fe₃S₄, and like materials.

By a superparamagnetic nanocluster is meant that the primary nanoparticles are present as clusters of primary nanoparticles, which clusters each comprise a more-or-less permanent shape and form (that is, a shape and form that remains intact during and after actions such as contacting the nanoclusters with a liquid sample, mixing or agitating the liquid sample (e.g. by ultrasonic agitation) and so on). Thus, a superparamagnetic nanocluster is comprised of primary (single-domain) nanoparticles (which may number e.g. in the tens, hundreds, thousands, or more) that are attached, e.g. bonded, together to form a stable structure. Such superparamagnetic nanoclusters can thus be distinguished from materials (such as ferrofluids and the like) that are made of primary nanoparticles that, even if they might temporarily aggregate or coalesce under certain conditions, are readily separable into individual primary nanoparticles, e.g. by ultrasonic agitation. The herein-disclosed superparamagnetic nanoclusters (which may range from e.g. 50 nanometers to about 1000 nanometers in overall diameter) may also be distinguished from permanently magnetic particles (even though some such permanently magnetic particles may be of similar size to some superparamagnetic nanoclusters). The herein-disclosed superparamagnetic nanoclusters may also be distinguished from primary nanoparticles that are e.g. embedded, encapsulated, or the like, at least partially within a layer or shell of organic polymeric material (e.g., polystyrene). In some particular embodiments, the herein-disclosed superparamagnetic nanoclusters do not comprise any portion (whether an interior or exterior layer, a partial coating, etc.) of organic polymeric material (e.g., polystyrene), excepting such organic polymers as carry carboxyl functional groups.

In specific embodiments, the superparamagnetic nanoclusters may comprise a diameter of at least about 30, 60, 100, 150, 200, 300, 400, or 500 nanometers. In further embodiments, the superparamagnetic nanoclusters may comprise a diameter of at most about 1000, 500, 400, or 200 nanometers.

By carboxyl-functionalized is meant that carboxyl functional groups are provided on the surfaces of the superparamagnetic nanoclusters (or of a tie layer thereon), in locations and conditions in which they are accessible by a liquid component of the liquid sample (e.g., in which the carboxyl groups are exposed so that they can be solvated by e.g. water molecules of a liquid sample). By carboxyl is meant —COOH groups, it being understood that such groups can exist in their neutral (—COOH) form, or can exist in their deprotonated (—COO⁻) form, depending e.g. on the pH of an aqueous environment in which the groups are placed. Carboxyl groups by definition do not include carbonyl groups of aldehydes, ketones, imides, urethanes, amides, or esters (unless such groups (e.g., certain esters) may be hydrolyzed to give rise to —COO⁻ groups when the nanoclusters are contacted with a liquid sample), particularly such carbonyl groups as might be present in conventional high molecular weight organic polymeric materials (e.g., polyesters, polyamides, and the like) that may be used to form beads, to provide coatings on beads, and so on. By carboxyl-functionalized is further meant that the carboxyl groups are present in a thin layer of about 2 nm or less in thickness, as discussed later herein.

As disclosed herein, the carboxyl groups remain at least substantially in place on the surface of the superparamagnetic nanoclusters during the contacting of the nanoclusters with the liquid sample and during the magnetic separation of the nanoclusters from the liquid sample (with the term substantially meaning that no more than about 10% of the carboxyl groups may be separated from the nanoclusters in such processes). In fact, such carboxyl functional groups may remain at least substantially in place during procedures such as washing of the superparamagnetic nanoclusters (which may be performed e.g. to remove unbound materials, reagents and the like, from the vicinity of the nanoclusters). In some embodiments at least some of the carboxyl groups may be covalently bound to the superparamagnetic nanoclusters. However, this is not strictly necessary, as long as the carboxyl groups are associated to the surface of the superparamagnetic nanoclusters strongly enough to remain in place during the herein-described processing.

In some embodiments, the superparamagnetic nanoclusters may inherently comprise carboxyl functional groups as a result of the process of synthesizing the nanoclusters. In some embodiments of this type, the carboxyl functional groups may be provided by a polymeric material (e.g., poly(meth)acrylic acid, sodium poly(meth)acrylate or the like) comprising carboxyl groups, which polymeric material is present in a reaction mixture that is used to synthesize the superparamagnetic nanoclusters and which polymeric material remains associated with (and possibly covalently bonded to) the synthesized superparamagnetic nanoclusters during the contacting and magnetically separating steps. In other embodiments of this type, the carboxyl functional groups may be provided by the polymerization of a monomeric or oligomeric material (e.g., sodium (meth)acrylate) comprising carboxyl groups, which monomeric or oligomeric material is present in a reaction mixture that is used to synthesize the superparamagnetic nanoclusters and which polymerizes during the synthesis of the superparamagnetic nanoclusters to form a polymeric material comprising carboxyl groups. The polymeric material remains associated with (and possibly covalently bonded to) the synthesized superparamagnetic nanoclusters during the contacting and magnetically separating steps.

In other embodiments, the carboxyl functional groups may be added to the surface of the superparamagnetic nanoclusters after the nanoclusters have been formed. For example, the carboxyl functional groups may be present as substituents on a monomeric, oligomeric, or high molecular polymeric material, that is contacted with the superparamagnetic nanoclusters so as to become associated with the surface thereof. In some particular embodiments, such a material may be covalently bonded to the nanoclusters (or to a material that is coated onto the surface of the superparamagnetic nanoclusters to facilitate this) as will be discussed in detail later herein.

The superparamagnetic nanoclusters may be synthesized using any suitable method. In some embodiments, the superparamagnetic nanoclusters may be synthesized by the general method known as high-temperature hydrolysis in which iron (e.g., iron (III)) cations are at least partially reduced at high temperature and are precipitated from solution to form nanoclusters of magnetite with primary particle sizes in appropriate size ranges. Such methods (which are described e.g. by Ge et. al., Chem. Eur. J. 2007, 13 (25), 7153-7161), may use e.g. polyacrylic acid as a capping agent, which polyacrylic acid may at least partially bind to the surfaces of the nanoparticles as they form and may remain at least partially bound thereto, and thus is ready-made to provide carboxylic acid functional groups for the purposes disclosed herein. In other embodiments, the superparamagnetic nanoclusters may be synthesized by the general method known as hydrothermal (sometimes referred to as solvothermal) synthesis in which a ferric precursor (e.g. ferric chloride hexahydrate) is dissolved in solution with various reagents (e.g. ethylene glycol and/or diethylene glycol, and sodium acrylate and/or sodium acetate), is heated and held at a high temperature for the reaction to proceed, and is then cooled to obtain the reaction product. In such methods (which are described e.g. by Xuan et. al., Chem. of Mat. 2009, 21, 5079-5087, which is incorporated by reference in its entirety herein for this purpose), sodium acrylate or the like may be used e.g. to confine and/or stabilize the growing nanoparticles e.g. to control the grain size thereof. It appears that the sodium acrylate polymerizes to at least some extent during the synthesis process (e.g., to form sodium polyacrylate); thus, this synthesis process may generate a carboxyl-functional oligomeric or polymeric material in situ, during the process of producing the superparamagnetic nanoclusters. Such carboxyl functional groups are then available for the purposes disclosed herein.

While the above-described methods may be particularly useful for the reasons mentioned herein, any suitable method of synthesis of superparamagnetic nanoclusters may be used (e.g., organometallic pyrolysis, chemical coprecipitation, micelle synthesis, laser pyrolysis), as long as the chosen method provides carboxyl functional groups or allows such groups to be associated with (e.g., attached to) the nanoclusters during or after synthesis of the nanoclusters.

In some embodiments, the carboxyl-functionalized superparamagnetic nanoclusters can be used as synthesized (and after any desired washing steps or the like are performed to remove reagents or raw materials). In other embodiments, at least a portion of the surfaces of at least some of the primary nanoparticles of the superparamagnetic nanoclusters can be coated with a material that may facilitate, or enhance, the association of carboxyl functional groups with the nanoclusters. Such a material may also serve to enhance the stability of the nanoclusters (i.e., it may enhance the ability of the primary nanoparticles from being unacceptably dislodged from the nanoclusters during the processing described herein), may serve to impart a more spherical shape to the nanoclusters, and so on. One exemplary material that has been found to serve all of these purposes is silica, which may be coated onto the superparamagnetic nanoclusters e.g. by a straightforward deposition process in which tetraethyl orthosilicate may be condensed (via hydrolysis) to form a layer of silica of desired thickness on some or all of the primary nanoparticles of the nanoclusters. In various embodiments, such a silica coating may comprise an average thickness of at least 2, 5, 10, 20, or 30 nanometers. In further embodiments, such a silica coating may comprise an average thickness of at most 100, 50, or 20 nanometers.

Such a silica coating can readily serve as a tie layer allowing any suitable molecules (comprising e.g. carboxyl functional groups) to be covalently bonded to the silica coating. For example, any suitable silanol-containing material such as e.g. carboxyethyl silane triol can be contacted with the silica surface so that the silane moieties react with surface hydroxyl groups of the silica to form covalent bonds, thus providing carboxyl groups that are tethered to the silica surface. In a variation of this, a so-called silane coupling agent (comprising e.g. a group that is readily convertible to a reactive silanol) can be contacted with the silica surface to similar effect. Thus, in particular embodiments, trimethoxysilyl propyl(ethylene-diamine triacetic acid) can be bonded to the silica surface via the trimethoxysilyl moieties (which hydrolyze in water to form silanols), leaving the three carboxyl groups of each molecule tethered to the silica surface. This provides a surface that will be referred to herein as comprising EDTA (ethylene-diamine tetraacetic acid)—derived carboxyl groups (noting that strictly speaking only three carboxyls are present on each molecule rather than four, since one carboxyl group was sacrificed to enable the silane coupling agent moiety to be bonded to the EDTA molecule).

If desired, a first, linker molecule (e.g. with a silane coupling agent at one end and with a suitable reactive group at the other end) can be attached to the nanoclusters (e.g., to the surface of a silica coating thereon) followed by the attachment of a second, carboxyl-containing molecule to the reactive group of the linker. However, such methods may be less convenient than e.g. the direct attachment of a carboxyl-containing molecule to the silica surface.

In summary, by such methods a tie layer of e.g. silica can be coated onto at least a portion of the surface of the superparamagnetic nanoclusters, and this coated layer of silica can then facilitate the attachment of carboxyl groups (whether in general, or in the specific form of EDTA-derived carboxyl groups) as well as providing other benefits as mentioned above.

Regardless of the particular method used to synthesize the superparamagnetic nanoclusters, all such superparamagnetic nanoclusters will be distinguished from certain conventional magnetic or superparamagnetic bead products in that the herein-disclosed superparamagnetic nanoclusters are not completely, or even partially, coated by, embedded within, and/or encapsulated by, any high molecular weight organic polymeric material (excepting such organic polymers as may carry the carboxyl functional groups). Thus, the disclosed superparamagnetic nanoclusters are distinguished from e.g. such beads as may comprise magnetic, paramagnetic, or superparamagnetic nanoparticles (or even nanoclusters) that are embedded, encased, etc., in polymeric materials such as polystyrene and the like. Furthermore, the herein-disclosed carboxyl groups (whether provided on a linker molecule such as provided e.g. by a silane coupling agent, or whether provided on an oligomer or polymer such as polyacrylic acid or the reaction product of an acrylate monomer or oligomer) by definition will be present on the nanoclusters (whether directly on the surface of the nanoparticles of the superparamagnetic nanoclusters or on the surface of a tie layer thereon) in a layer of about 5 nanometers or less in average thickness. The presence of carboxyl groups in such a thin layer will distinguish the herein-disclosed carboxyl-functionalized superparamagnetic nanoclusters from e.g. products in which magnetic, paramagnetic, or superparamagnetic particles are embedded partially or completely within a relatively thick shell of e.g. carboxyl-containing polymeric materials. In various embodiments, the average thickness of the carboxyl-containing layer of the herein-disclosed superparamagnetic nanoparticles may be less than about 2, 1.5, or 1 nanometers. In further embodiments, the average thickness of the carboxyl-containing layer of the herein-disclosed superparamagnetic nanoparticles may be at least about 0.2, 0.5, or 1 nanometers.

In various embodiments, the nanoclusters may comprise a superparamagnetic material content of at least about 50, 70, 80, or 90% by weight (with the balance being comprised of the carboxyl groups (and any e.g. oligomeric or polymeric material that the carboxyl groups are on) and silica (if present)). In specific embodiments, the nanoclusters may comprise an iron oxide content of at least about 50, 70, 80, or 90% by weight.

It is further noted that many magnetic beads or particles known in the art (even those functionalized with carboxyl groups), are characterized as exhibiting low non-specific binding (e.g., of proteins). Thus, the ordinary artisan would not expect such beads or particles to exhibit the ability to non-specifically bind microorganisms that is documented in the Working Examples herein. Rather, most such carboxyl groups appear to be provided e.g. for the chelation of metal ions, or to facilitate the attachment to the beads of particular moieties (e.g., antibodies and the like) that can provide specific binding to particular microorganism strains.

The carboxyl-functionalized superparamagnetic nanoclusters can be used in any form that is amenable to contacting the nanoclusters with a liquid sample. For example, the nanoclusters may be used in particulate form (e.g., in a carrier liquid, for example as a suspension or dispersion) or applied to a support such as a dipstick, film, filter, tube, well, plate, beads, membrane, or channel of a microfluidic device, or the like.

The term “liquid” sample is used broadly to encompass not only liquids and liquid solutions, but also any sample in which one or more solid or semi-solid materials is present (e.g., suspended, dispersed, emulsified, etc.) in a liquid (noting further that such a solid material does not necessarily have to be stably suspended in the liquid). Nor does the liquid necessarily have to exhibit a particularly low viscosity (thus, a liquid sample could be a slurry, a filter cake, or the like, as long as sufficient liquid is present to allow the herein-disclosed process to be performed). Often, the liquid sample may be an aqueous sample in which liquid water makes up a significant portion (e.g., at least 20, 40, 60, 80, 90, or 95% by weight) of the liquid sample.

The processes disclosed herein can be applied to a variety of different types of liquid samples, including, but not limited to, medical, environmental, food, feed, clinical, and laboratory samples, and combinations thereof. Medical or veterinary samples can include, for example, cells, tissues, or fluids from a biological source. Environmental samples can be, for example, from a medical or veterinary facility, an industrial facility, soil, a water source, a food preparation area (food contact and non-contact areas), a laboratory, or an area that has been potentially subjected to bioterrorism. Food processing, handling, and preparation area samples are preferred, as these are often of particular concern in regard to food supply contamination by bacterial pathogens.

Such samples can be used directly, or can be concentrated (for example, by centrifugation) or diluted (for example, by the addition of a buffer (pH-controlled) solution) prior to the process being performed. Samples in the form of a solid or a semi-solid can be used directly or can be extracted, if desired, by a method such as, for example, washing or rinsing with, or suspending or dispersing in, a fluid medium (for example, a buffer solution). Samples can be taken from surfaces (for example, by swabbing or rinsing). Examples of samples that can be used include foods, beverages, potable water, water used in any biochemical or industrial process, and biological fluids (for example, whole blood or a component thereof), cell preparations (for example, dispersed tissue, bone marrow aspirates, or vertebral body bone marrow); cell suspensions; urine, saliva, and other body fluids, as well as lysed preparations, which can be formed using known procedures such as the use of lysing buffers, and the like. Preferred samples include foods, beverages, potable water, biological fluids, and combinations thereof. In particular embodiments, the liquid sample may be a complex semi-solid mixture derived from one or more foods (e.g., a slurry obtained from grinding one or more solid or semi-solid foods into a liquid).

The term “microorganism” is used broadly to denote any cell having genetic and/or proteomic material suitable for analysis or detection. The term also encompasses any fragment, portion, remnant, residue, etc., of such a microorganism, that can provide evidence that the microorganism had been present (whether intact or in fragments) in the liquid sample. Such fragments might include, but are not limited to, e.g. cell walls and portions thereof. The term “strain” means a particular type of microorganism that may be distinguished through a detection method (for example, microorganisms of different genera, of different species within a genus, or of different isolates or strains within a species). It is noted however that the methods disclosed herein are devoted to non-specific separation of any such microorganism strains from a liquid sample, and moreover the methods do not necessarily require that any particular strain ever be identified as such.

Microorganisms that can be separated from a liquid sample using the methods disclosed herein include, for example, bacteria, fungi, yeasts, protozoans, viruses, and the like, and combinations thereof. The process has utility in the detection of pathogens, which can be important for food safety or for medical, environmental, or anti-terrorism reasons. The process can be particularly useful in the detection of pathogenic bacteria (for example, both gram negative and gram positive bacteria, or combinations thereof), as well as various yeasts, molds, and mycoplasmas.

Genera of target microorganisms to be separated include, but are not limited to, Listeria, Escherichia, Salmonella, Campylobacter, Clostridium, Helicobacter, Mycobacterium, Staphylococcus, Shigella, Enterococcus, Bacillus, Neisseria, Shigella, Streptococcus, Vibrio, Yersinia, Bordetella, Borrelia, Pseudomonas, Saccharomyces, Candida, and the like, and combinations thereof. Specific microorganism strains include Escherichia coli O157: H7, Yersinia enterocolitica, Yersinia pseudotuberculosis, Vibrio cholerae, Vibrio parahaemolyticus, Vibrio vulnificus, Listeria monocytogenes, Staphylococcus aureus, Salmonella enterica, Saccharomyces cerevisiae, Candida albicans, Bacillus cereus, Bacillus anthracia, Clostridium perfringens, Clostridium botulinum, Clostridium difficile, and the like, and combinations thereof.

Separation of any such microorganisms using the methods disclosed herein is generally not specific to any particular strain, species, or type of microorganism and therefore provides for the separation, from a liquid sample, of a general population of microorganisms in the sample. Thus, such microorganisms can be concentrated from the level at which they were present in the liquid sample. If desired, specific strains of microorganisms can then be detected from among the separated microorganism population using any known detection method e.g. with strain-specific probes. Thus, the methods disclosed herein can be used e.g. for the detection of microbial contaminants or pathogens (particularly food-borne pathogens such as bacteria) in clinical, food, environmental, or other samples.

Any suitable method of providing contact between the superparamagnetic nanoclusters and the liquid sample can be used. For example, the superparamagnetic nanoclusters (whether e.g. alone, in a carrier liquid, or on a suitable carrier or support matrix) can be added to the liquid sample, or vice versa. A dipstick coated with nanoclusters can be immersed in a liquid sample, a liquid sample can be poured onto a film coated with nanoclusters, a liquid sample can be poured into a tube or well coated with nanoclusters, or a liquid sample can be passed through a filter (for example, a woven or nonwoven filter) coated with nanoclusters. It may be particularly convenient that the superparamagnetic nanoclusters and the liquid sample are combined (using any order of addition) in any of a variety of containers (optionally but preferably, a capped, closed, or sealed container; more preferably, a capped test tube, bottle, or jar). Suitable containers will be determined by the particular sample and can vary widely in size and nature. Mixing and/or agitation (for example, by stirring, shaking, vortexing, or use of a rocking platform) and/or any another process that facilitates bringing the nanoclusters, and any microorganisms in the liquid sample, into close proximity to each other so that non-specific binding can take place, may be used as desired. If desired, one or more additives (for example, lysis reagents, nucleic acid capture reagents, microbial growth media, buffers (for example, to moisten a solid sample), microbial staining reagents, washing buffers (for example, to wash away unbound material), elution agents (for example, serum albumin), surfactants (for example, Triton™ X-100 nonionic surfactant available from Union Carbide Chemicals and Plastics, Houston, Tex.), mechanical abrasion/elution agents (for example, glass beads), and the like) can be included in the combination of the nanoclusters and the liquid sample.

The superparamagnetic nanoclusters may be held in contact with the liquid sample for any suitable time, and under any suitable conditions, to allow non-specific binding to occur between the microorganisms and the carboxyl groups of the nanoclusters. While not wishing to be limited by theory or mechanism, it may be that such non-specific binding occurs partly, or even primarily, by way of non-specific interactions between the carboxyl groups of the nanoclusters, and proteins (or protein fragments) that may be present on the cell walls (or fragments thereof) of any microorganisms that may be present in the liquid sample. Without wishing to be limited by theory or mechanism, such non-specific binding between e.g. the carboxyl groups of the nanoclusters and microorganisms or fragments thereof, might involve e.g. electrostatic interaction (including but not limited to hydrogen bonding), hydrophobic interaction, or of any combination thereof. Regardless of what form it takes, this non-specific binding by definition does not encompass any kind of specific interaction, affinity, or binding (e.g., antigen-antibody, enzyme-substrate, or receptor-ligand binding, binding between complementary nucleic acids, binding between avidin or streptavidin and biotin, or the like).

The process as disclosed herein further comprises separating of at least some of the nanoclusters from the liquid sample, along with any microorganisms that are non-specifically bound to the nanoclusters. It will be appreciated that some small amount of liquid will typically remain in contact with the separated nanoclusters (and associated microorganisms if present). It is thus emphasized that complete separation of all of the nanoclusters from the entirety of the liquid sample is not necessarily required; that is, the methods disclosed herein may range from achieving e.g. generally or substantially complete separation of the nanoclusters (and any microorganisms bound thereto) from the liquid sample, to merely imparting a desired concentrating effect of the microorganisms in the liquid sample.

After appropriate contacting time and conditions to allow sufficient binding to occur between the superparamagnetic nanoclusters and microorganisms (if present), a magnetic force may be applied to separate at least some of the superparamagnetic nanoclusters from at least some of the liquid. Such magnetic separation may take the form of using magnetic force to move at least some of the superparamagnetic nanoclusters through the liquid. Or, magnetic force can be used to hold at least some of the superparamagnetic nanoclusters in place while at least some of the liquid (e.g. supernatant) of the liquid sample is moved away from the nanoclusters (for example, by decanting or siphoning, so as to leave the nanoclusters at or near a surface of the container as held there by the magnetic force). Any desired combination of these two approaches may be used. Any suitable permanent magnet or electromagnet, or multiple magnets or combinations thereof, may be used. Any such magnet(s) may be held stationary relative to the liquid sample, or may be moved relative to the liquid sample, during the separating process. Such processes can be carried out manually (for example, in a batch-wise manner) or can be automated (for example, to enable continuous or semi-continuous processing). Any other separation method (e.g., centrifugation, filtration, etc.) may also be used in conjunction with the magnetic separating (e.g., either before, during or after the magnetic separating) to enhance the separation achieved by the superparamagnetic nanoclusters.

As evidenced by the Working Examples herein, the disclosed superparamagnetic nanoclusters have been found to be surprisingly effective in binding microorganisms such as e.g. bacteria so that the microorganisms can be separated from a liquid sample, even though the nanoclusters do not comprise any moiety that is capable of performing specific bonding to any such microorganism. That is, the disclosed superparamagnetic nanoclusters do not comprise any type of e.g. affinity binding group, antibody or antigen, etc., and are thus believed to achieve the described separation by non-specific binding achieved e.g. by way of the carboxyl groups. Thus, in specific embodiments, the nanoclusters do not comprise any substituent capable of specifically binding to the at least one microorganism strain. That is, in such embodiments the nanoclusters do not comprise any antibody, antigen, template, affinity group, complementary nucleic acid or the like, that is configured to bind with a specific group of a specific target microorganism or portion or fragment thereof. In other words, the fact that the disclosed carboxyl-functionalized superparamagnetic nanoclusters may, under some conditions, exhibit superior ability to capture certain microorganism strains, versus certain other microorganism strains, cannot be taken to mean that the nanoclusters perform specific binding of any such microorganisms.

In general, it is noted that the superparamagnetic nanoclusters disclosed herein may e.g. achieve at least generally similar, or even shorter, separation times in comparison to other magnetic materials (of any type), while not sacrificing the ability to capture microorganisms with acceptable efficiency. That is, the disclosed superparamagnetic nanoclusters may exhibit generally similar, or even superior, performance to other magnetic materials in terms of e.g. Capture Efficiency.

After the separating process, the carboxyl-functionalized superparamagnetic nanoclusters can be assayed in order to detect evidence of one or more microorganism strains that are (or were, at least up through the conclusion of the magnetic separating process) non-specifically bound thereto. That is, such assaying may reveal whether or not the liquid sample contained a detectable level any such microorganisms. It is emphasized that not every performing of the method will necessarily reveal that a detectable level of microorganisms were present in the liquid sample tested. That is, many samples (e.g., of potable water and the like) may be tested with negative results (that is, with the result that the level of microorganisms in the sample appeared to be below a particular detection threshold).

The assaying can be performed by any suitable detection method. (One or more washing steps, and/or other steps as desired, may be performed following the magnetic separating of the nanoclusters from the liquid sample, either prior to, or as part of the subsequent assaying operation). Suitable detection methods might include, for example, microscopy (for example, using a transmitted light microscope or an epifluorescence microscope, which can be used for visualizing microorganisms tagged with fluorescent dyes) and other imaging methods, immunological detection methods, and genetic detection methods. Immunological detection is detection of an antigenic material derived from a target organism, which is commonly a biological molecule (for example, a protein or proteoglycan) acting as a marker on the surface of bacteria or viral particles. Detection of the antigenic material typically can be by an antibody, a polypeptide selected from a process such as phage display, or an aptamer from a screening process. Immunological detection methods are well-known and include, for example, immunoprecipitation and enzyme-linked immunosorbent assay (ELISA). Antibody binding can be detected in a variety of ways (for example, by labeling either a primary or a secondary antibody with a fluorescent dye, with a quantum dot, or with an enzyme that can produce chemiluminescence or a colored substrate, and using either a plate reader or a lateral flow device).

Detection can also be carried out by genetic assay (for example, by nucleic acid hybridization or primer directed amplification), which is often a preferred method. The captured or bound microorganisms can be lysed to render their genetic material available for assay. Lysis methods are well-known and include, for example, treatments such as sonication, osmotic shock, high temperature treatment (for example, from about 50° C. to about 100° C.), and incubation with an enzyme such as lysozyme, glucolase, zymolose, lyticase, proteinase K, proteinase E, and viral enolysins. Many commonly-used genetic detection assays detect the nucleic acids of a specific microorganism, including the DNA and/or RNA. Particularly useful genetic detection methods are based on primer directed nucleic acid amplification (for example, polymerase chain reaction (PCR), real-time PCR, reverse transcriptase polymerase chain reaction (RT-PCR), and ligase chain reaction (LCR)), as well as isothermal methods and strand displacement amplification (SDA) (and combinations thereof; preferably, PCR or RT-PCR)).

Since use of the carboxyl-functionalized superparamagnetic particles as disclosed herein is non-strain specific, it can provide a general separation system that may allow for multiple microorganism strains to be targeted for detection in the same liquid sample. For example, in assaying for contamination of food samples, it can be desired to test for Listeria monocytogenes, E. coli O157:H7 and Salmonella all in the same sample. A single capture step can then be followed by, for example, PCR or RT-PCR assays using specific primers to amplify different nucleic acid sequences from each of these microorganism strains. Thus, the need for separate sample handling and preparation procedures for each strain can be avoided.

Thus, in some embodiments, rather than assaying for the presence of a specific strain or strains, a general method can be used that may allow detection of any such microorganisms. One such method may involve e.g. contacting the magnetically-separated superparamagnetic nanoclusters with a liquid sample, separating the superparamagnetic nanoclusters from the liquid sample, optionally exposing the nanoclusters to e.g. a lysing agent to disrupt any cells present to allow their contents to be exposed, and then inspecting the e.g. lysed sample for the presence of ATP (adenosine triphosphate). (The nanoclusters may or may not be magnetically separated from the lysed sample before the ATP inspection is performed, as desired.) Inspection of such a sample might be performed e.g. by bioluminescence. Such methods will be appreciated as revealing the presence of most any e.g. plant or animal microorganism (since all such microorganisms use ATP for metabolic functioning); thus, such methods may provide a useful, non-specific screening test for the presence of microorganisms in general. Other such methods may be culture-based methods which might comprise e.g. plating the magnetically-separated superparamagnetic nanoclusters onto growth media, culturing the growth media, and determining the presence and/or number of bacterial colonies growing on the growth media. Such methods may again be performed for e.g. non-specific screening; or, one or more types of microorganisms, or specific microorganism strains, may be targeted (e.g., by providing growth media that is specifically targeted to promote the growth of colonies of certain microorganism strains).

A kit for use in carrying out the processes described herein may be provided. Such a kit may contain any carboxyl-functionalized superparamagnetic nanoclusters as disclosed herein, in any suitable form in which the nanoclusters can be contacted with a liquid sample. Ancillary equipment and supplies such as reagents, diluents, containers, stirring instruments, and so on, may of course be supplied with such a kit. Such a kit might also comprise one or more components selected from microorganism culture or growth media, lysis reagents, buffers, genetic detection assay components, and so on. One or more magnets may be supplied with the kit; or, such magnets may be kept on hand by a user and used with a succession of kits. Such a kit may comprise instructions for carrying out the process of claim 1 (noting that such a kit specifically includes a virtual kit, in which such instructions are provided electronically rather than in paper form).

### LIST OF EXEMPLARY EMBODIMENTS

### Embodiment 1

A process comprising: contacting a plurality of carboxyl-functionalized superparamagnetic nanoclusters with a liquid sample potentially comprising at least one microorganism strain; magnetically separating at least some of the carboxyl-functionalized superparamagnetic nanoclusters from at least a portion of the liquid sample; and assaying the magnetically-separated superparamagnetic nanoclusters for evidence of the at least one microorganism strain having been non-specifically bound thereto.

### Embodiment 2

The process of embodiment 1, wherein the superparamagnetic nanoclusters comprise high-temperature-hydrolysis-synthesized superparamagnetic nanoclusters.

### Embodiment 3

The process of embodiment 1, wherein the superparamagnetic nanoclusters comprise hydrothermally-synthesized superparamagnetic nanoclusters.

### Embodiment 4

The process of any of embodiments 1-3, wherein at least some of the superparamagnetic nanoclusters inherently comprise accessible carboxyl functional groups on the surfaces of the nanoclusters as a result of the synthesis process.

### Embodiment 5

The process of embodiment 4, wherein the carboxyl functional groups of the carboxyl-functionalized superparamagnetic nanoclusters are provided by a polymeric material comprising carboxyl groups, which polymeric material is provided in a reaction mixture that is used to synthesize the superparamagnetic nanoclusters and which polymeric material remains associated with the synthesized superparamagnetic nanoclusters during the magnetically separating and assaying steps.

### Embodiment 6

The process of embodiment 4, wherein the carboxyl functional groups of the carboxyl-functionalized superparamagnetic nanoclusters are provided by the polymerization of a monomeric or oligomeric material comprising carboxyl groups, which monomeric or oligomeric material is provided in a reaction mixture that is used to synthesize the superparamagnetic nanoclusters and polymerizes during the synthesis of the superparamagnetic nanoclusters to form a polymeric material comprising carboxyl groups, which polymeric material remains associated with the synthesized superparamagnetic nanoclusters during the magnetically separating and assaying steps.

### Embodiment 7

The process of embodiment 6, wherein the carboxyl functional groups are the reaction product of the polymerization of sodium acrylate.

### Embodiment 8

The process of any of embodiments 6-7, wherein the plurality of carboxyl-functionalized superparamagnetic nanoclusters comprises silica-coated superparamagnetic nanoclusters in which the surfaces of the silica coatings have been functionalized with carboxyl groups.

### Embodiment 9

The process of embodiment 8, wherein the carboxyl groups are EDTA-derived carboxyl groups that are on molecules that are covalently bonded to the surfaces of the silica coatings.

### Embodiment 10

The process of embodiment 9, wherein the molecules that are covalently bonded to the surfaces of the silica coatings are the reaction product of N-(trimethyoxysilylpropyl)ethylene-diamine triacetic acid with hydroxyl groups of the silica coatings.

### Embodiment 11

The process of any of embodiments 1-10 wherein the liquid sample is an aqueous sample.

### Embodiment 12

The process of any of embodiments 1-11 wherein the liquid sample is a complex semi-solid mixture derived from one or more foods.

### Embodiment 13

The process of any of embodiments 1-12 wherein the at least one microorganism strain is a bacteria strain.

### Embodiment 14

The process of any of embodiments 1-13 wherein the at least one microorganism strain comprises an E. coli strain.

### Embodiment 15

The process of any of embodiments 1-13 wherein the at least one microorganism strain comprises a Listeria monocytogenes strain.

### Embodiment 16

The process of any of embodiments 1-15, wherein the assaying of the magnetically-separated superparamagnetic nanoclusters for evidence of the at least one microorganism strain having been non-specifically bound thereto, is carried out by a method selected from culture-based methods, microscopy and other imaging methods, genetic detection methods, immunologic detection methods, and combinations thereof.

### Embodiment 17

The process of any of embodiments 1-16 wherein the assaying of the magnetically-separated superparamagnetic nanoclusters for evidence of the at least one microorganism strain having been non-specifically bound thereto, comprises disposing the magnetically-separated superparamagnetic nanoclusters onto a medium and inspecting the medium for the presence of ATP.

### Embodiment 18

The process of any of embodiments 1-16 wherein the assaying of the magnetically-separated superparamagnetic nanoclusters for evidence of the at least one microorganism strain having been non-specifically bound thereto, comprises plating the magnetically-separated superparamagnetic nanoclusters onto a growth media, culturing the growth media, and determining the presence, absence, or number, of bacterial colonies growing on the growth media.

### Embodiment 19

The process of any of embodiments 1-18 wherein the superparamagnetic nanoclusters do not comprise any substituent capable of specifically binding to any specific microorganism strain.

### Embodiment 20

The process of any of embodiments 1-19 wherein the superparamagnetic nanoclusters collectively exhibit an average diameter of from about 50 to about 200 nanometer, and wherein each superparamagnetic nanocluster comprises a collection of single-domain nanoparticles of magnetite of from about 5 to about 20 nanometer in average diameter.

### Embodiment 21

The process of any of embodiments 1-20 wherein the superparamagnetic nanoclusters remain substantially intact and the carboxyl functional groups thereof remain substantially in place on the superparamagnetic nanoclusters, during the contacting of the superparamagnetic nanoclusters with the liquid sample and during the magnetically separating of the superparamagnetic nanoclusters from at least a portion of the liquid sample.

### Embodiment 22

The process of any of embodiments 1-21 wherein the superparamagnetic nanoclusters are not at least partially coated by, embedded within, and/or encapsulated by, any high molecular weight non-polar organic polymeric material.

### Embodiment 23

A kit comprising the plurality of carboxyl-functionalized superparamagnetic nanoclusters of any of embodiments 1-22 and comprising instructions for carrying out at least the process of embodiment 1.

### EXAMPLES

### Preparation of Superparamagnetic Magnetite Nanoclusters

Materials:

Diethylene glycol (DEG, reagent grade) was purchased from Fisher Scientific (Pittsburgh, Pa.). Anhydrous iron(III) chloride (FeCl3, 98%) was purchased from Strem Chemicals (Newburyport, Mass.). Polyacrylic acid (PAA, Mw=1800), sodium hydroxide (NaOH, 99.9%), iron(III) chloride hexahydrate (FeCl₃.6H₂O, 97%), sodium acrylate, sodium acetate, ethylene glycol (anhydrous, 99.8%) sodium oleate (99%), and tetraethyl orthosilicate (TEOS, 98%) were purchased from Sigma-Aldrich (St. Louis, Mo.). Ethyl alcohol (anhydrous) and ammonia hydroxide (NH₄OH, 30%) were purchased from EMD Chemicals (Billerica, Mass.). N-(trimethoxysilylpropyl)ethylenediamine triacetate, trisodium (TMS-EDTA, 45% in water) was purchased from Gelest (Morrisville, Pa.).

Sample A

Superparamagnetic magnetite (Fe₃O₄) nanoclusters were synthesized by high-temperature hydrolysis method according to literature procedures (Ge et. al., Chem. Eur. J. 2007, 13 (25), 7153-7161) with small modifications. A NaOH/DEG stock solution was prepared by dissolving 2 g of NaOH in 20 mL of DEG. This solution was heated at 120° C. for 1 hour under nitrogen, and then cooled down to 70° C. In a three-neck flask, a mixture of 0.288 g of PAA, 17 mL of DEG, and 0.065 g of anhydrous FeCl₃ were heated to 220° C. for 30 minutes under nitrogen and vigorous stirring. Then, 2.0 mL of NaOH/DEG stock solution was rapidly injected into the above hot mixture. This reaction solution was further heated for 1 hour at 210° C. and then cooled down to room temperature. The synthesized magnetite nanoclusters were precipitated out by adding 40 mL of ethanol and centrifugation. The precipitates were redispersed in 5 mL of DI water and the nanoclusters were then collected by magnet after adding 20 mL of ethanol. Then, the nanoclusters were washed several times by precipitation with ethanol and redispersion in deionized water. Finally, the magnetite nanoclusters were dispersed in deionized water at 4 mg/ml concentration.

Sample B

Superparamagnetic magnetite (Fe₃O₄) nanoclusters were synthesized by hydrothermal method according to literature procedures (Xuan et. al., Chem. of Mat. 2009, 21, 5079-5087) with Small modifications. 0.54 g of FeCl₃.6H₂O, 1.5 g of sodium acrylate, 1.5 g of sodium acetate, 5 mL of ethylene glycol, and 15 mL of diethylene glycol were mixed together under magnetic stirring for 2 hours. The obtained homogeneous solution was transferred into a Teflon®-lined stainless-steel reaction vessel and heated at 200° C. for 15 hours. The synthesized magnetite nanoclusters were precipitated out by adding 40 mL of ethanol and centrifugation. The precipitates were redispersed in 5 mL of DI water and the nanoclusters were then collected by magnet after adding 20 mL of ethanol. Then, the nanoclusters were washed several times by precipitation with ethanol and redispersion in deionized water. Finally, the magnetite nanoclusters were dispersed in DI water at 10 mg/ml concentration.

Sample C

Superparamagnetic magnetite (Fe₃O₄) nanoclusters were synthesized in generally similar manner to the nanoclusters of Sample B. The obtained nanoclusters (150 mg) were redispersed in 10 mL of DI water. 20 mg of Na oleate was dissolved in 5 mL of DI water by heating it at 70° C. Then Fe₃O₄ nanocluster dispersion was added dropwise into Na oleate solution and stirred for 30 minutes. The particles were washed twice using DI water and redispersed in deionized water at 10 mg/ml concentration.

Sample D

Superparamagnetic magnetite (Fe₃O₄) nanoclusters were synthesized in generally similar manner to the nanoclusters of Sample B, with the addition of the following steps. 100 mg of Fe₃O₄ nanoclusters were redispersed in 12 mL of DI water and further diluted with 120 mL of ethanol. 4 mL of NH₄OH was added into the above solution and agitated in ultrasonic bath for 15 minutes. Then, 0.6 mL of TEOS in 5 mL of ethanol was added and agitated in ultrasonic bath for 2 hours. The core-shell particles was separated out from the solution by magnet and washed twice by DI water. 100 mg of TMS-EDTA was added into the core-shell particle dispersion, which was heated at 85° C. for 16 hours. The EDTA grafted core-shell particles were washed twice using DI water and redispersed in DI water at a concentration of 10 mg/mL.

Characterization of Samples

The sizes of the superparamagnetic nanoclusters of Samples A-D were characterized by using Hitachi H-9000 transmission electron microscope (TEM) operated at 300 kV. Samples were diluted at a rough ratio of 20 drops sample to 20 mL of water. Diluted samples were sonicated for 15 minutes, and a single drop of the sonicated and diluted sample was placed on an ultrathin carbon TEM grid and allowed to dry in air. Estimated sizes were taken from these TEM images. These images also confirmed each nanocluster was an aggregate of many primary nanoparticles. It did not appear that the nanoclusters fragmented into individual primary nanoparticles, or otherwise decreased in size to any significant extent, in routine handling in liquid media.

The estimated diameters of the nanoclusters of Samples A-D as obtained from these measurements are listed in Table 1 along with the vendor-supplied nominal sizes of the following two Comparative Sample Materials:

Comparative Sample CS-A: Magnetic particles (nominal 100 nm diameter) comprising polyacrylic acid were purchased from Chemicell Inc. (Berlin, Germany) under the trade designation fluid MAG-PAS.

Comparative Sample CS-B: Magnetic particles (nominal 1000 nm diameter) reported to be coated with carboxylic acid were purchased from Invitrogen (Oslo, Norway) under the trade designation Dynabeads MyOne Carboxylic Acid.

TABLE 1

| Sample ID | Size (nm) |
|---|---|
| A | 90 |
| B | 60 |
| C | 100 |
| D | 150 |
| CS-A | 100 |
| CS-B | 1000 |

The stability of the carboxyl functional groups on the surface of the nanoclusters of Samples A and B was demonstrated by measuring the FTIR spectrum. FTIR spectra were acquired with a Nicolet 6700 Series FT-IR spectrometer using a single-reflection Pike SmartMIRacle germanium ATR accessory and a DTGS detector at 4 cm-1 resolution. FTIR spectra of Samples A and B were taken, along with Comparative Sample CS-A. All three materials exhibited peaks located at ˜1560 cm-1 and ˜1405 cm-1 that appeared to be indicative of asymmetric and symmetric C—O stretching modes of the carboxyl group. The spectra remained relatively stable even after extensive washing with DI water; thus, it appears that the carboxyl groups were stably associated with the surfaces of the nanoclusters.

### Microbiological Performance Evaluation of Samples

Materials:

Stocks of bacterial cultures of Escherichia coli (E. coli) (ATCC 51813) and Listeria monocytogenes (ATCC 51414) were obtained from ATCC (American Type Culture Collection, Manassas, Va.). Laboratory plastic supplies and reagents and bacterial culture media were believed to be from VWR unless otherwise stated. Specific materials and their source are listed below in Table 2.

Working Samples A-D were all sonicated for approximately 5 minutes using a benchtop sonication unit before use, to ensure that the materials were well-dispersed. Comparative Sample materials CS-A and CS-B were vortexed for approximately 10 seconds with a benchtop vortex mixer prior to use.

TABLE 2

| Material | Source |
|---|---|
| Butterfield's | pH 7.2 ± 0.2, monobasic potassium phosphate buffer |
| buffer (BBL) | solution, obtained from VWR, West Chester, PA (VWR |
| | Catalog Number 83008-093) |
| DI water | Deionized, filtered, 18 megaohm water, processed through |
| | Milli-Q Gradient System obtained from Millipore; |
| | Waltham, MA |
| E. coli plate | E. coli detection plate obtained 3M Company, St. Paul, |
| | MN, under the trade designation “3M E. COLI/COLIFORM |
| | PETRIFILM PLATE” |
| MOX plate | Plate with Oxford Medium, modified for Listeria, obtained |
| | from Hardy Diagnostics, Santa Maria, CA |
| Stomacher | Laboratory blender, obtained from VWR under the trade |
| | designation “STOMACHER 400 CIRCULATOR |
| | LABORATORY BLENDER” |
| Stomacher | Polyethylene sample bags, obtained from VWR under the |
| bags | trade designation “FILTRA-BAG” (VWR Catalog |
| | #89085-574) |
| Tryptic Soy | Plate with DIFCO Tryptic Soy Agar obtained from BD, |
| Agar (TSA) | Sparks, MD, prepared at 3% according to the manufactur- |
| plate | er's instructions |
| Tryptic Soy | Tryptic soy broth from Becton Dickinson, Sparks, MD, |
| Broth (TSB) | prepared at 3% concentration according to the manufactur- |
| | er's instructions |
| 0.5 | McFarland standards are used as a reference to adjust the |
| McFarland | turbidity of bacterial suspensions so that the number of |
| Standard | bacteria will be within a given range. A 0.5 McFarland |
| | number corresponds to bacterial concentration of about |
| | 1-1.5 × 10⁸ CFU/mL. Turbidity was adjusted using a |
| | densitometer (obtained from bioMerieux, Inc., Durham, |
| | NC, under the trade designation “DENSICHECK“) |

Separation Time

Ground beef was purchased from local grocery store (Cub Foods, St. Paul, Minn.). 11 grams of ground beef (15% fat) was added a sterile Stomacher bag and blended with 99 ml Butterfield's Buffer solution in a Stomacher 400 Circulator laboratory blender for a 30 second cycle at 230 rpm speed to generate a blended ground beef sample.

A 1 ml volume of the beef sample was added to a sterile 1.5 ml polypropylene microcentrifuge tube. One mg of Sample C was added to the tube. The tube was capped and was manually inverted for about 10 seconds. The tube was put on a magnetic stand (Dynal MPC-L, Invitrogen, Oslo, Norway) and separation time of the sample (as evident by visual observation) was noted. Sample D, and Comparative Examples CS-A and CS-B, were prepared and tested similarly. The observed separation times are listed in Table 3.

TABLE 3

| Example No. | Sample ID | Separation Time (secs) |
|---|---|---|
| 1 | C | 15 |
| 2 | D | 30 |
| CE-1 | CS-A | 60 |
| CE-2 | CS-B | 45 |

Capture of L. monocytogenes from Ground Beef

A blended ground beef sample was prepared as described above. A single colony of Listeria monocytogenes from an overnight streak plate culture on a TSA plate was inoculated into 10 ml of TSB and incubated at 37° C. in a shaker incubator (Innova44 from New Brunswick Scientific) for 18-20 hrs. The resulting bacterial stock containing ˜1×10⁹ CFU/mL was serially diluted in BBL to obtain an approximately 1×10⁵ CFU/mL inoculum “Listeria microorganism suspension”, which was inoculated in the blended ground beef sample to obtain a “spiked beef sample” at 1×10³ CFU/mL. (Here and elsewhere, CFU means Colony Forming Units.) The material was used in the following experiments.

### Example 3

A 1.0 mL volume of the “spiked beef sample” was added to a labeled, sterile 5 mL polypropylene tube polypropylene tube (here and elsewhere, obtained from Becton Dickinson, Franklin Lakes, N.J., under the trade designation “BD FALCON”) containing 100 microliters (from a 4 mg/ml stock) of Sample A.

### Example 4

A 1.0 mL volume of the “spiked beef sample” was added to a labeled, sterile 5 mL polypropylene tube containing 200 microliters (from a 4 mg/ml stock) of Sample A.

### Example 5

A 1.0 mL volume of the “spiked beef sample” was added to a labeled, sterile 5 mL polypropylene tube polypropylene tube containing 100 microliters (from a 10 mg/ml stock) of Sample C.

### Example 6

A 1.0 mL volume of the “spiked beef sample” was added to a labeled, sterile 5 mL polypropylene tube containing 100 microliters (from a 10 mg/ml stock) of Sample D.

### Comparative Example CE-3

A 1.0 mL volume of the “spiked beef sample” was added to a labeled, sterile 5 mL polypropylene tube containing 100 microliters (from a 50 mg/ml stock) of Comparative Sample CS-A. Comparative Examples CE-4 and CE-5 were prepared and tested in the same manner as CE-3, except that a 1.0 mL volume of the “spiked beef sample” was added to 40 microliters (from a 25 mg/ml stock), and to 200 microliters, of Comparative Sample CS-A (versus 100 microliters in example CE-3 and in the Working Examples) in Examples CE-4 and CE-5 respectively.

### Comparative Example CE-6

A 1.0 mL volume of the “spiked beef sample” was added to a labeled, sterile 5 mL polypropylene tube containing 100 microliters/1 mg (from a 10 mg/ml stock) of Comparative Sample CS-B.

The tubes were capped and kept on a rocking platform (Thermolyne Vari Mix rocking platform (Barnstead International, Iowa, 14 cycles/minute) for a contact time of 10 minutes after which the superparamagnetic nanoclusters (or Comparative Sample beads) were separated using a magnetic stand (Dynal MPC-L, Invitrogen, Oslo, Norway) for about 2.5 minutes. The supernatant liquid was removed from each tube (by pipetting, while the superparamagnetic nanoclusters were held (by the magnetic force) against the surface of the tube closest to the external magnet) and the magnetically-separated materials were resuspended in 100 microliters BBL and plated on MOX plates. 100 microliter aliquots from each supernatant liquid sample were also plated on MOX plates. (This enabled the % Capture achieved by the magnetic materials to be ascertained by subtraction, since in many cases the number of cells captured by the magnetic materials were so high as to give a “Too Numerous to Count” result when the magnetically captured materials were plated).

The various plates were incubated at 37° C. for 18-20 hours and manually analyzed for colony counts. As stated above, confluent growth of >100 CFU/mL (also known as “Too Numerous To Count”) often resulted from plating of the resuspended magnetic materials on MOX plates. Therefore, capture efficiency was calculated by the alternative procedure of obtaining colony counts from plating the remaining liquid sample that resulted after removal of the magnetic materials (with appropriate correction based on plated unconcentrated control samples). These calculations were performed as follows:

% Control=(Colony counts from plated remaining sample/Colony counts from unconcentrated control sample)×100

Capture Efficiency or % Capture=100−% Control

Results in % Capture are reported in Table 4. (The unconcentrated control had an average colony count of 3765 CFU/mL, excepting Example CE-4 which was tested in a separate assay where the unconcentrated control had an average colony count of 2370 CFU/mL.)

TABLE 4

| Example No. | Sample ID | Capture Efficiency (%) |
|---|---|---|
| 3 | A | 60 |
| 4 | A | 75 |
| 5 | C | 46 |
| 6 | D | 97 |
| CE-3 | CS-A | 82 |
| CE-4 | CS-A | 97 |
| CE-5 | CS-A | 79 |
| CE-6 | CS-B | 16 |

Capture of E. coli from Water (Assaying by Culturing)

An overnight streaked culture of Escherichia coli from a TSA plate (incubated at 37° C.) was used to make a 0.5 McFarland Standard in 3 ml filtered distilled deionized water. The resulting bacterial stock containing 1×10⁸ CFU/mL was serially diluted in water to obtain an approximately 1×10⁵ CFU/mL “E. coli microorganism suspension”, which was used in the following experiments.

### Example 7

A 1.0 mL volume of the E. coli microorganism suspension was added to a labeled, sterile 5 mL polypropylene tube containing 250 microliters (from a 4 mg/ml stock) of Sample A.

### Example 8

A 1.0 mL volume of the E. coli microorganism suspension was added to a labeled, sterile 5 mL polypropylene tube containing 100 microliters (from a 10 mg/ml stock) of Sample B.

### Comparative Example CE-7

A 1.0 mL volume of the E. coli microorganism suspension was added to a labeled, sterile 5 mL polypropylene tube containing 20 microliters (from a 50 mg/ml stock) of Comparative Sample CS-A.

### Comparative Example CE-8

A 1.0 mL volume of the E. coli microorganism suspension was added to a labeled, sterile 5 mL polypropylene tube containing 100 microliters (from a 10 mg/ml stock) of Comparative Sample CS-B.

The tubes were sealed with parafilm and vortexed for 10 seconds to mix. The tubes were kept on a rocking platform (Thermolyne Vari Mix rocking platform (Barnstead International, Iowa, 14 cycles/minute) for a contact time of 10 minutes after which the superparamagnetic nanoclusters (or Comparative Sample beads) were magnetically separated using a magnetic stand (Dynal MPC-L, Invitrogen, Oslo, Norway) for about 2.5 minutes. The sample was removed and the magnetically-separated materials were resuspended in 1 ml BBL and plated on E. coli plates. (Typically, the supernatant liquid was also plated so that % Capture could be calculated therefrom, in the manner described above). The plates were incubated at 37° C. for 18-20 hours and analyzed for colony counts per manufacturer's instructions using a Petrifilm Plate reader (3M Company, St. Paul, Minn.).

Capture efficiency was calculated based on colony counts obtained from the plated remaining sample and plated unconcentrated control sample by using the formulas below.

% Control=(Colony counts from plated remaining sample/Colony counts from unconcentrated control sample)×100

Capture Efficiency or % Capture=100−% Control

Results are reported in Table 5. (The unconcentrated control had an average colony count of 185,000 CFU/mL.)

TABLE 5

| Example No. | Sample ID | Capture Efficiency (%) |
|---|---|---|
| 7 | A | 31 |
| 8 | B | 75 |
| CE-7 | CS-A | 0 |
| CE-8 | CS-B | 4 |

Capture of E. coli from Water (Assaying by ATP)

Examples 9, 10, and CE-9 and CE-10 were prepared in similar manner as Examples 7, 8, and CE-7 and CE-8, and were kept on a rocking platform and then separated using a magnetic stand as described above. After this, the separated materials were resuspended in 50 microliters of an extractant (lysis) solution and 500 microliters of an enzyme solution from a sample preparation kit (obtained from 3M Company; St. Paul, Minn., under the trade designation “3M CLEAN-TRACE SURFACE ATP SYSTEM”). The contents of the tube were mixed by vortexing for about 15 seconds, at about 3200 rpm on a vortex mixer (obtained from VWR, West Chester Pa., under the trade designation “VWR FIXED SPEED VORTEX MIXER”). After this, the superparamagnetic nanoclusters (or Comparative Sample beads) were separated on a magnetic stand. That is, for each sample, while the magnetic beads were held by magnetic force against the surface of the tube closest to the external magnet, the supernatant liquid was removed via pipette and was then added to a sterile 1.5 ml polypropylene microcentrifuge tube (VWR, Catalog #89000-028).

The ATP signal of each such sample was measured in relative light units (RLU) for one minute at 10 second intervals using a bench-top luminometer (obtained from Turner Biosystems, Sunnyvale, Calif., under the trade designation “20/20N SINGLE TUBE LUMINOMETER”, equipped with 20/20n SIS software). Luminescence values were analyzed as described below.

The background ATP level of each magnetic material was determined by adding the same volume of ATP reagents to same volumes of materials as the test samples, but without any E. coli being present. These background values were subtracted from the ATP signals from the test samples to calculate the “Corrected ATP Signal” values as shown in Table 6. The ATP signal measured for 100 microliters water containing 10⁵ CFU was used as a “10⁵ ATP Signal Control”. The % ATP signal was calculated from the Corrected ATP Signal values for the controls according to the following equation:

% ATP Signal=(Corrected RLU/RLU from 10⁵ CFU Control)×100

Results are shown in Table 6. Also, it was noted that Example CE-9 (*) exhibited a much higher standard deviation in the ATP signal than did the other Examples.

TABLE 6

| | | | | % ATP |
|---|---|---|---|---|
| | | | Corrected | Signal of |
| | | ATP Signal | ATP Signal | 10⁵ CFU |
| Example No. | Sample ID | (RLU) | (RLU) | control |
| — | (ATP Signal | 25578 | | |
| | Control) | | | |
| 9 | A | 23082 | 23001 | 90 |
| 10 | B | 9124 | 8093 | 35 |
| CE-9 | CS-A | 2699* | 2624 | 29 |
| CE-10 | CS-B | 642 | 428 | 16 |

The foregoing Examples have been provided for clarity of understanding only. No unnecessary limitations are to be understood therefrom. The tests and test results described in the Examples are intended solely to be illustrative, rather than predictive, and variations in the testing procedure can be expected to yield different results. All quantitative values in the Examples are understood to be approximate in view of the commonly known tolerances involved in the procedures used.

It will be apparent to those skilled in the art that the specific exemplary structures, features, details, configurations, etc., that are disclosed herein can be modified and/or combined in numerous embodiments. (In particular, all elements that are positively recited in this specification as alternatives, may be explicitly included in the claims or excluded from the claims, in any combination as desired.) All such variations and combinations are contemplated by the inventor as being within the bounds of the conceived invention not merely those representative designs that were chosen to serve as exemplary illustrations. Thus, the scope of the present invention should not be limited to the specific illustrative structures described herein, but rather extends at least to the structures described by the language of the claims, and the equivalents of those structures. To the extent that there is a conflict or discrepancy between this specification as written and the disclosure in any document incorporated by reference herein, this specification as written will control.

What is claimed is:

1. A process comprising:
contacting a plurality of carboxyl-functionalized superparamagnetic nanoclusters with a liquid sample potentially comprising at least one microorganism strain;
magnetically separating at least some of the carboxyl-functionalized superparamagnetic nanoclusters from at least a portion of the liquid sample; and
assaying the magnetically-separated superparamagnetic nanoclusters for evidence of the at least one microorganism strain having been non-specifically bound thereto.

2. The process of claim 1, wherein the superparamagnetic nanoclusters comprise high-temperature-hydrolysis-synthesized superparamagnetic nanoclusters.

3. The process of claim 1, wherein the superparamagnetic nanoclusters comprise hydrothermally-synthesized superparamagnetic nanoclusters.

4. The process of claim 1, wherein at least some of the superparamagnetic nanoclusters inherently comprise accessible carboxyl functional groups on the surfaces of the nanoclusters as a result of the synthesis process.

5. The process of claim 4, wherein the carboxyl functional groups of the carboxyl-functionalized superparamagnetic nanoclusters are provided by a polymeric material comprising carboxyl groups, which polymeric material is provided in a reaction mixture that is used to synthesize the superparamagnetic nanoclusters and which polymeric material remains associated with the synthesized superparamagnetic nanoclusters during the magnetically separating and assaying steps.

6. The process of claim 4, wherein the carboxyl functional groups of the carboxyl-functionalized superparamagnetic nanoclusters are provided by the polymerization of a monomeric or oligomeric material comprising carboxyl groups, which monomeric or oligomeric material is provided in a reaction mixture that is used to synthesize the superparamagnetic nanoclusters and polymerizes during the synthesis of the superparamagnetic nanoclusters to form a polymeric material comprising carboxyl groups, which polymeric material remains associated with the synthesized superparamagnetic nanoclusters during the magnetically separating and assaying steps.

7. The process of claim 6, wherein the carboxyl functional groups are the reaction product of the polymerization of sodium acrylate.

8. The process of claim 6, wherein the plurality of carboxyl-functionalized superparamagnetic nanoclusters comprises silica-coated superparamagnetic nanoclusters in which the surfaces of the silica coatings have been functionalized with carboxyl groups.

9. The process of claim 8, wherein the carboxyl groups are EDTA-derived carboxyl groups that are on molecules that are covalently bonded to the surfaces of the silica coatings.

10. The process of claim 9, wherein the molecules that are covalently bonded to the surfaces of the silica coatings are the reaction product of N-(trimethyoxysilylpropyl)ethylene-diamine triacetic acid with hydroxyl groups of the silica coatings.

11. The process of claim 1 wherein the liquid sample is an aqueous sample.

12. The process of claim 1 wherein the liquid sample is a complex semi-solid mixture derived from one or more foods.

13. The process of claim 1 wherein the at least one microorganism strain is a bacteria strain.

14. The process of claim 1 wherein the at least one microorganism strain is an E. coli strain.

15. The process of claim 1 wherein the at least one microorganism strain is a Listeria monocytogenes strain.

16. The process of claim 1, wherein the assaying of the magnetically-separated superparamagnetic nanoclusters for evidence of the at least one microorganism strain having been non-specifically bound thereto, is carried out by a method selected from culture-based methods, microscopy and other imaging methods, genetic detection methods, immunologic detection methods, and combinations thereof.

17. The process of claim 1 wherein the assaying of the magnetically-separated superparamagnetic nanoclusters for evidence of the at least one microorganism strain having been non-specifically bound thereto, comprises disposing the magnetically-separated superparamagnetic nanoclusters onto a medium and inspecting the medium for the presence of ATP.

18. The process of claim 1 wherein the assaying of the magnetically-separated superparamagnetic nanoclusters for evidence of the at least one microorganism strain having been non-specifically bound thereto, comprises plating the magnetically-separated superparamagnetic nanoclusters onto a growth media, culturing the growth media, and determining the presence, absence, or number, of bacterial colonies growing on the growth media.

19. The process of claim 1 wherein the superparamagnetic nanoclusters do not comprise any substituent capable of specifically binding to any specific microorganism strain.

20. The process of claim 1 wherein the superparamagnetic nanoclusters collectively exhibit an average diameter of from about 50 to about 200 nanometer, and wherein each superparamagnetic nanocluster comprises a collection of single-domain nanoparticles of magnetite of from about 5 to about 20 nanometer in average diameter.

21. The process of claim 1 wherein the superparamagnetic nanoclusters remain substantially intact and the carboxyl functional groups thereof remain substantially in place on the superparamagnetic nanoclusters, during the contacting of the superparamagnetic nanoclusters with the liquid sample and during the magnetically separating of the superparamagnetic nanoclusters from at least a portion of the liquid sample.

22. The process of claim 1 wherein the superparamagnetic nanoclusters are not at least partially coated by, embedded within, and/or encapsulated by, any high molecular weight non-polar organic polymeric material.

23. A kit comprising the plurality of carboxyl-functionalized superparamagnetic nanoclusters of claim 1 and comprising instructions for carrying out the process of claim 1.
